(19) United States
(12) Patent Application Publication
(10) Pub. No.: US 2024/0338040 A1
(43) Pub. Date: Oct. 10, 2024

(54) ROBOTICALLY MANIPULATED SENSORS FOR AGRICULTURAL HABITATS

(71) Applicant: Sensei Ag Holdings, Inc., Santa Monica, CA (US)

(72) Inventors: W. Daniel Hillis, Santa Monica, CA (US); David Douglas, Santa Monica, CA (US)

(21) Appl. No.: 18/627,275

(22) Filed: Apr. 4, 2024

Related U.S. Application Data
(60) Provisional application No. 63/494,953, filed on Apr. 7, 2023.

(51) Int. Cl. G05D 1/667; G01J 3/28; G01N 33/00; G05D 105/20; G05D 105/80; G05D 107/20

(52) U.S. Cl. CPC G05D 1/667; G01J 3/28; G01N 33/0098; G01J 2003/283; G05D 2105/20; G05D 2105/80; G05D 2107/21

(57) ABSTRACT

A sensor module having multiple sensors for collecting, analyzing, storing, and transmitting data related to environmental and plant growth data in an agricultural habitat, such as a greenhouse, is disclosed. One or more modules are placed at various locations in a greenhouse, to collect data including any of temperature, temperature gradient, humidity, light levels, light frequencies, soil moisture, soil composition, plant health, plant growth, plant quality e.g. maturity and/or fruit quantity and/or ripeness. The sensor module is adapted for robotic placement within the greenhouse, although the module ay initially be placed by hand. The module is powered by internal batteries or a large capacitor or capacitor array and is recharged by a greenhouse robot that may comprise any of a robot arm configured for movement within a greenhouse via a conveyer or track system, a wheeled robot, or a drone.

### CROSS-REFERENCE TO RELATED APPLICATION(S)

This application claims priority to provisional patent application Ser. No. 63/494,953, filed Apr. 7, 2023, which application is incorporated herein in its entirety by this reference thereto.

### TECHNICAL FIELD

Various of the disclosed embodiments concern robotically manipulated sensors for agricultural habitats.

### BACKGROUND

Greenhouses and other agricultural habitats must provide a nurturing environment for the plants that are grown within them. For example, a greenhouse must maintain an appropriate and stable ambient temperature if the plants in the greenhouse are to thrive. However, given the wide range of environmental conditions found in greenhouses, such as variations in ambient temperature and thermal build up, e.g. the greenhouse effect, monitoring environmental factors such as temperature, light, and humidity, as well as plant development and health, in a greenhouse or other agricultural habitat is important.

### SUMMARY

Embodiments of the invention provide a sensor module having multiple sensors for collecting, analyzing, storing, and wirelessly transmitting data related to environmental and plant growth data in an agricultural habitat such as a greenhouse environment. In embodiments of the invention, one or more modules are placed at various locations in a greenhouse, e.g. between plant rows, within plant beds, at different elevations within the greenhouse, etc., to collect data including any of temperature, temperature gradient, humidity, light levels, light frequencies, soil moisture, soil composition, plant health, plant growth, plant quality, e.g. maturity and/or fruit quantity and/or ripeness.

In embodiments of the invention, the sensor module is adapted for robotic placement within the greenhouse, although the module may initially be placed by hand. The robotic system is used to position the sensor module. Each sensor module may have a regular location within the greenhouse or the robotic system may regularly reposition the sensor modules within the greenhouse based on any of a predetermined schedule and/or upon information derived from one or more sensors modules.

The module is adapted to be powered by internal batteries or a large capacitor or capacitor array. The module is recharged by the robotic system, for example by engaging a robot appendage with the sensor module to make an electrical connection with charging contacts or by coupling with charging inductors within the sensor module.

The robotic system may comprise any of a robot arm configured for movement within a greenhouse via a conveyer or track system, a wheeled robot, or a drone.

### BRIEF DESCRIPTION OF THE DRAWINGS

FIG. 1 is a perspective schematic view showing a greenhouse with one or more sensor modules placed therein, as well as the robot that moves and charges the sensor modules, according to the invention;

FIG. 2 is a block schematic diagram that shows a sensor module for an agricultural habitat according to an embodiment of the invention;

FIG. 3 shows a perspective view of a sensor module for an agricultural habitat according to an embodiment of the invention; and

FIG. 4 is a block diagram of a computer system as may be used to implement certain features of some of the embodiments.

### DETAILED DESCRIPTION

Embodiments of the invention provide a rechargeable module having multiple sensors for collecting, analyzing, storing, and wirelessly transmitting data related to environmental and plant growth data in an agricultural habitat such as a greenhouse environment. In embodiments of the invention one or more modules are placed at various locations in a greenhouse, e.g. between plant rows, within plant beds, at different elevations within the greenhouse, etc., to collect data including any of temperature, temperature gradient, humidity, light levels, light frequencies, soil moisture, soil composition, plant health, plant growth, plant quality, e.g. maturity, health, and/or fruit quantity and/or ripeness.

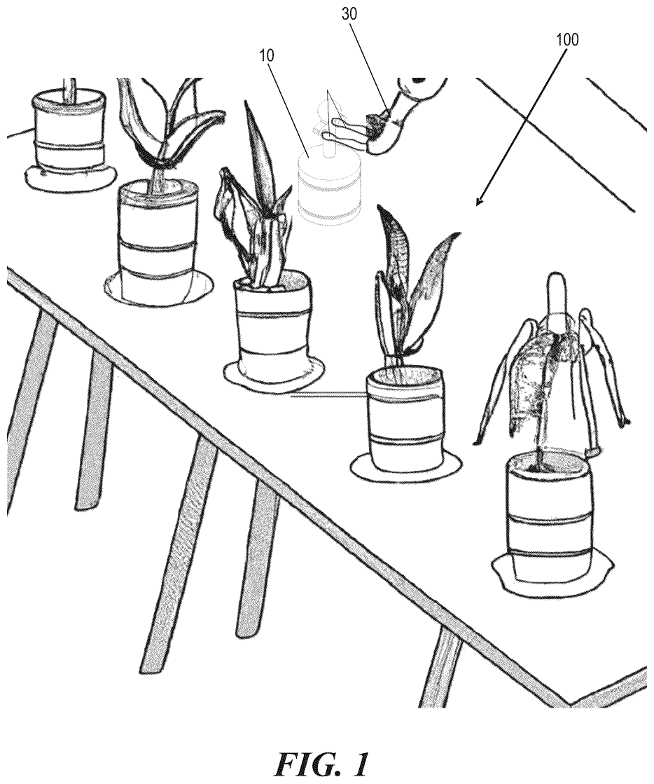

FIG. 1 is a perspective schematic view showing a greenhouse with one or more sensor modules placed therein, as well as the robot that moves and charges the sensor modules, according to the invention.

In embodiments of the invention the sensor module 10 is adapted for robotic placement within a greenhouse 100, although the module may initially be placed by hand. The robotic system 30 is used to position the sensor module. Each sensor module may have a regular location within the greenhouse or the robotic system may regularly reposition the sensor modules within the greenhouse based on any of a predetermined schedule and/or upon information derived from one or more sensor modules. For example, sensor data may be collected at each of several locations; the information thus collected is then mapped to each location and associated with a group of plants at that location. If the progress of the plants in different locations is not uniform, the sensors can be repositioned to identify conditions at those locations where growth was less than that desired. Thus, by monitoring multiple locations substandard greenhouse infrastructure can be identified and mitigated.

The module is adapted to be powered by internal batteries or a large capacitor or capacitor array. The module is recharged by the robotic system, for example by engaging a robot appendage with the sensor module to make an electrical connection with charging contacts or by coupling with charging inductors within the sensor module.

The robotic system may comprise any of a robot arm configured for movement within a greenhouse via a conveyer or track system, a wheeled robot, or a drone.

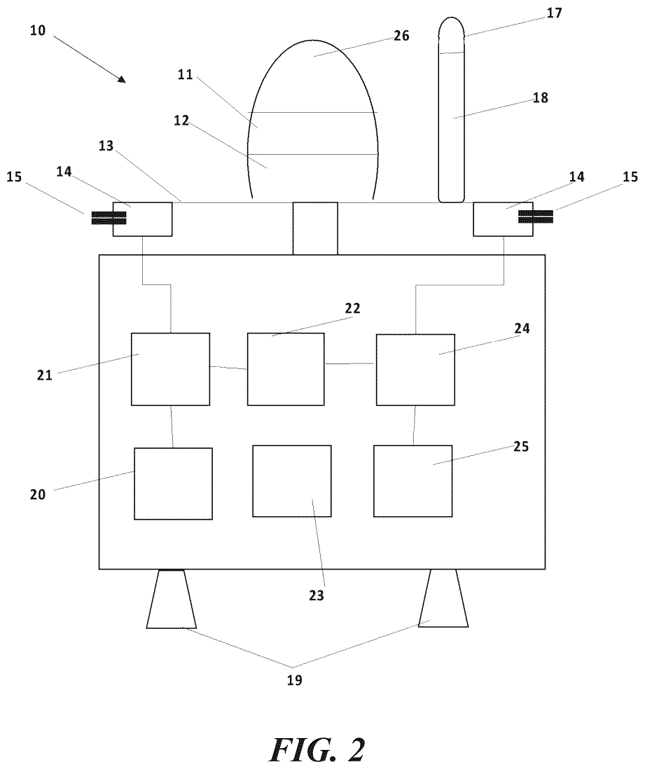

FIG. 2 is a block schematic diagram that shows a sensor module for an agricultural habitat according to an embodiment of the invention.

In FIG. 2, a sensor module 10 includes a plurality of sensors such as a light sensor 26, wind sensor 11, pressure sensor 12, and temperature, CO2, and humidity sensors 23. The light sensor may include one or more filters to measure the various wavelengths of light to which the plants are exposed. The external system may adjust light exposure times and/or wavelengths based on this information. The filters may be automatically exchangeable over predetermined intervals such that a single set of sensors may be used to measure several wavelengths of light.

The sensor module may also incorporate a camera to monitor plant growth and health. Those skilled in the art will appreciate that other or additional sensors, e.g. pH sensors, may be included in the sensor module as appropriate for the application to which the sensor module is put. For example, sensors on the sensor module may be configured to come into direct contact with the soil in which the plants are growing to identify percentages of soil components, e.g. potassium, nitrogen, and/or phosphorus.

Information collected by the sensors is provided to an electronic assembly 21 and then processed by a processor 25. The sensor module can store this information in a RAM or EPROM memory 22 and manipulate the information if programed to do so. For example, the processor can run a program that identifies environmental trends within the greenhouse and then prepares a report for export from the sensor module to external systems, such as a greenhouse control system. The greenhouse control system then adjusts such parameters as light, humidity, water, etc. accordingly.

In embodiments of the invention, the sensor module communicates with external systems via a wireless modem. Such communications can use any known protocol, for example W-Fi, Bluetooth, G5, etc. The sensor module transmits sensor information to, and receives control information from, external systems. An antenna 18 is provided for the wireless modem. Information from several sensor modules may be exported to an external system that maps such information to various beds within the greenhouse based on sensor module location. Such information can be used to identify locations within the greenhouse which are more or less conducive to healthy growth. Growing conditions at location that are less productive can then be adjusted and plant growth can be monitored until those locations also provide optimal growing conditions.

An optical beacon 17 identifies the position of the sensor module and may also be used to signal to a robotic system such information as a need for energy, the need to upload data to an external system, a detected greenhouse problem, such as excessive temperature or unhealthy plants, or to identify that there is a system fault within the sensor module itself. The color of the beacon may indicate status, e.g. operational, fault, charge needed. Further, each sensor module may incorporate a unique label, such as a bar code or QR code, that may be used to identify the sensor module. For example, a camera in the robotic system or in a drone could scan the label and use the sensor module identity to map the location of the module within the greenhouse.

The sensor module is intended to be positioned and repositioned within a growing environment such as a greenhouse. As such, the sensor module includes lift rings 14 with which the sensor module may be picked up and moved, for example by a robot arm or drone. In other embodiments of the invention, the sensor module may include a ferromagnetic plate that allows the sensor module to be lifted and moved by a magnet or electromagnet in the robot system.

In embodiments of the invention, the lift rings include an insulated mount that has electrical contacts 15 by which the sensor module may receive power to charge the energy store 20 and by which the sensor module may be interrogated to receive or output information. For example, the electrical contacts may be used to upload sensor data that is stored in the sensor module for collection by an external system.

Energy may be supplied to the sensor module to charge the energy store by photocells 13 that are incorporated into the sensor module structure. In other embodiments, an inductive charging mechanism may be incorporated within the sensor module and the robotic system would include a complementary inductive system for imparting a charge to the sensor module via the charging inductors within the sensor module.

The sensor module sits on feet 19 when set down. This maintains a stable support for the sensor module, where the sensor module is not always set on a level surface, e.g. when placed in a bed of plants. The feet may include actuators and a leveling system to allow the sensor module to be automatically leveled once placed.

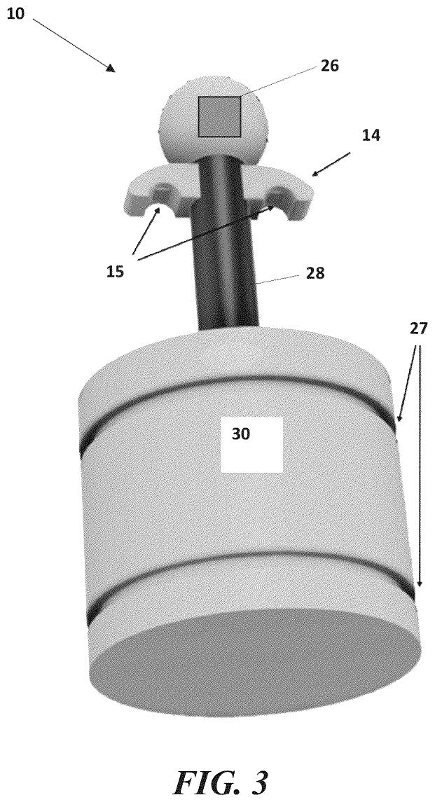

FIG. 3 shows a perspective view of a sensor module for an agricultural habitat according to an embodiment of the invention.

As shown in FIG. 3, the sensor module 10 includes sensors, such as a light sensor 26. Power, sensors, and electronics 30 are held within a container. The container is vented 27. An antenna mast and lift handle 28 projects upwardly from the container. Lift tabs 14 and charging contacts 15 are arranged on the antenna mast and lift handle.

The sensor module may be made of any material suitable for a greenhouse environment. This includes various metals and plastic materials. Further, while FIG. 2 shows a sensor module having the shape of a flowerpot, those skilled in the art will appreciate that the sensor module may be produced in any desired shape, i.e. any shape that can be lifted by a robot.

### Computer Implementation

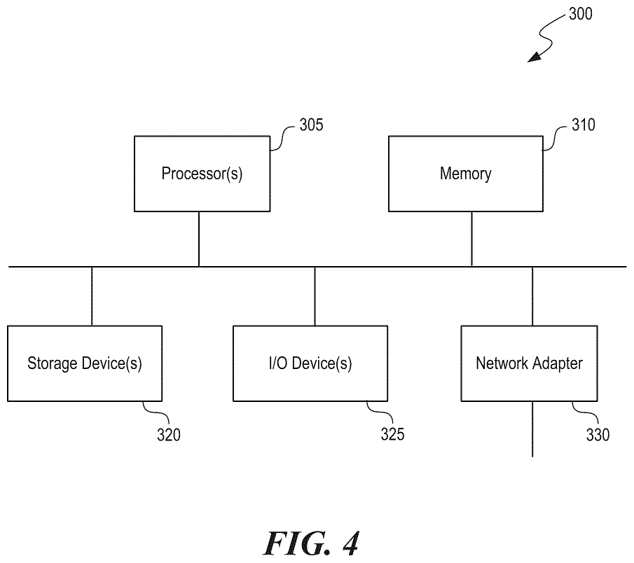

FIG. 4 is a block diagram of a computer system as may be used to implement certain features of some of the embodiments. The computer system may be a server computer, a client computer, a personal computer (PC), a user device, a tablet PC, a laptop computer, a personal digital assistant (PDA), a cellular telephone, an iPhone, an iPad, a processor, a telephone, a web appliance, a network router, switch or bridge, a console, a hand-held console, a (hand-held) gaming device, a music player, any portable, mobile, hand-held device, wearable device, or any machine capable of executing a set of instructions (sequential or otherwise) that specify actions to be taken by that machine.

The computing system 300 may include one or more central processing units (“processors”) 305, memory 310, input/output devices 325, e.g. keyboard and pointing devices, touch devices, display devices, storage devices 320, e.g. disk drives, and network adapters 330, e.g. network interfaces, that are connected to an interconnect 315. The interconnect 315 is illustrated as an abstraction that represents any one or more separate physical buses, point to point connections, or both connected by appropriate bridges, adapters, or controllers. The interconnect 315, therefore, may include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (12C) bus, or an Institute of Electrical and Electronics Engineers (IEEE) standard 1394 bus, also called Firewire.

The memory 310 and storage devices 320 arc computer-readable storage media that may store instructions that implement at least portions of the various embodiments. In addition, the data structures and message structures may be stored or transmitted via a data transmission medium, e.g. a signal on a communications link. Various communications links may be used, e.g. the Internet, a local area network, a wide area network, or a point-to-point dial-up connection. Thus, computer readable media can include computer-readable storage media, e.g. non-transitory media, and computer-readable transmission media.

The instructions stored in memory 310 can be implemented as software and/or firmware to program the processor 305 to carry out actions described above. In some embodiments, such software or firmware may be initially provided to the processing system 300 by downloading it from a remote system through the computing system 300, e.g. via network adapter 330.

The various embodiments introduced herein can be implemented by, for example, programmable circuitry, e.g. one or more microprocessors, programmed with software and/or firmware, or entirely in special-purpose hardwired (non-programmable) circuitry, or in a combination of such forms. Special-purpose hardwired circuitry may be in the form of, for example, one or more ASICs, PLDs, FPGAs, etc.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

I/We claim:

1. An apparatus, comprising:
one or more sensor modules adapted to be placed at one or more selected locations within an agricultural habitat;
said one or more sensor modules comprising:
an energy source;
a plurality of individual sensors adapted to collect data comprising any two or more of temperature, temperature gradient, humidity, light levels, light frequencies, soil moisture, soil composition, plant health, plant growth, and plant quality including any of maturity, health, fruit quantity, and ripeness;
a processor for processing and manipulating information collected by said plurality of sensors and storing said information in a memory, wherein said processor identifies environmental trends within the agricultural habitat and prepares a report of said trends for export from said sensor module; and
a communications medium by which sensor module transmits sensor information to, and receives control information from, external systems.

2. The apparatus of claim 1, wherein said selected locations comprise any of:
between plant rows;
within plant beds; and
at different elevations within the agricultural habitat.

3. The apparatus of claim 1, further comprising:
a robotic system adapted to periodically place and reposition at least one of said one or more sensor modules within the agricultural habitat based on any of a predetermined schedule and/or upon information derived from one or more of said sensor modules.

4. The apparatus of claim 3, wherein said one or more sensor modules are adapted for placement by said robotic system at said one or more selected locations within said agricultural habitat.

5. The apparatus of claim 1, said one or more sensor modules further adapted to collect data at each of several locations within the agricultural habitat; and
said one or more sensor modules further adapted to map said collected data to each location and associate said collected data with a group of plants at said location.

6. The apparatus of claim 5, said one or more of said sensor modules further adapted to identify conditions at locations among said several locations where growth is not acceptable.

7. The apparatus of claim 5, wherein said one or more of said sensor modules are repositioned when growth of the plants at said several locations is not uniform.

8. The apparatus of claim 5, said one or more of said sensor modules further adapted to monitor multiple locations to identify and mitigate substandard agricultural habitat infrastructure.

9. The apparatus of claim 1, said energy source of said one or more of said sensor modules comprising any of internal batteries and a large capacitor or capacitor array.

10. The apparatus of claim 1, said energy source of said one or more of said sensor modules further comprising a power source that is adapted to be recharged by either of electrical connection with charging contacts or inductive coupling with charging inductors by engagement with an appendage of a robotic system.

11. The apparatus of claim 3, said robotic system comprising any of a robot arm configured for movement within said agricultural habitat via a conveyer or track system, a wheeled robot, or a drone.

12. The apparatus of claim 1, said plurality of sensors comprising any of a light sensor, a wind sensor, a pressure sensor, a temperature sensor, a CO2 sensor, a humidity sensor, a pH sensor, and an imaging device to monitor plant growth and health.

13. The apparatus of claim 12, said light sensor further comprising one or more filters adapted to measure different wavelengths of light to which plants within the agricultural habitat are exposed.

14. The apparatus of claim 13, said one or more filters adapted to be automatically exchangeable at associated light sensors over predetermined intervals, wherein said associated light sensors each measure different wavelengths of light.

15. The apparatus of claim 5, said light sensor further comprising an external system configured to adjust light exposure times and/or wavelengths of light applied to plants within said agricultural habitat based on said mapping.

16. The apparatus of claim 1, said plurality of sensors comprising a sensor configured for direct contact with a medium in which plants in the agricultural habitat are grown to identify percentages of medium components.

17. The apparatus of claim 1, said one or more sensor modules further configured to export information to an external system that maps said information to different beds within the agricultural habitat based on sensor module location; and
said external system using said information to identify locations within the agricultural habitat that are more or less conducive to healthy plant growth.

18. The apparatus of claim 1, said one or more sensor modules further comprising:
a beacon configured for any of:
identifying a sensor module position; and
signaling to a robotic system sensor module status comprising any of a need for energy, a need to upload data to an external system, a detected greenhouse problem, and to identify a system fault within the sensor module itself.

19. The apparatus of claim 18, wherein a color of the beacon indicates said status.

20. The apparatus of claim 1, said one or more sensor modules further comprising:
a unique label to identify the sensor module.

21. The apparatus of claim 20, wherein an imaging device in a robotic system or in a drone scans the label and uses the sensor module identity to map a location of the module within the agricultural habitat.

22. The apparatus of claim 1, said one or more sensor modules further configured to be positioned and repositioned within the agricultural habitat by either of lift rings or a ferromagnetic plate with which the sensor module may be picked up and moved by a robot system.

23. The apparatus of claim 22, said lift rings further comprising:
an insulated mount having either of electrical contacts or an inductive coupling mechanism by which the sensor module receives power to charge said energy source and by which the sensor module may be interrogated to receive information from, or output information to, an external system.

24. The apparatus of claim 1, said one or more sensor modules further comprising:
one or more photocells incorporated into the sensor module structure to charge the energy source within the sensor module.

25. The apparatus of claim 1, said one or more sensor modules further comprising:
one or more feet configured to maintain a stable support for the sensor module, wherein said one or more feet comprise actuators and a leveling system to automatically level the sensor module after it is placed.

26. A method, comprising:
placing one or more sensor modules at one or more selected locations within an agricultural habitat;
said one or more sensor modules:
collecting with one or more sensors data comprising any two or more of temperature, temperature gradient, humidity, light levels, light frequencies, soil moisture, soil composition, plant health, plant growth, and plant quality including any of maturity, health, fruit quantity, and ripeness;
processing and manipulating information collected by said sensors and storing said information in a memory;
identifying environmental trends within the agricultural habitat and preparing a report of said trends for export from said sensor module; and
transmitting sensor information to, and receiving control information from, external systems.

27. The method of claim 26, further comprising:
with a robotic system periodically placing and repositioning at least one of said one or more sensor modules within the agricultural habitat based on any of a predetermined schedule and/or upon information derived from one or more of said sensor modules.

28. The method of claim 27, further comprising:
collecting data at each of several locations within the agricultural habitat;
mapping said collected data to each location; and
associating said collected data with a group of plants at said location.

29. The method of claim 28, further comprising:
identifying conditions at locations among said several locations where growth is not acceptable.

30. The method of claim 28, further comprising:
repositioning one or more of said sensor modules when growth of the plants at said several locations is not uniform.

31. The method of claim 28, further comprising:
monitoring multiple locations to identify and mitigate substandard agricultural habitat infrastructure.

32. The method of claim 26, wherein said plurality of sensors comprise any of a light sensor, a wind sensor, a pressure sensor, a temperature sensor, a CO2 sensor, a humidity sensor, a pH sensor, and an imaging device to monitor plant growth and health.

33. The method of claim 26, further comprising:
measuring with one or more filters different wavelengths of light to which plants within the agricultural habitat are exposed.

34. The method of claim 33, further comprising:
automatically exchanging said one or more filters at associated light sensors over predetermined intervals, wherein said associated light sensors each measure different wavelengths of light.

35. The method of claim 28, further comprising:
an external system adjusting light exposure times and/or wavelengths of light applied to plants within said agricultural habitat based on said mapping.

36. The method of claim 26, further comprising:
exporting information to an external system that maps said information to different beds within the agricultural habitat based on sensor module location; and
said external system using said information to identify locations within the agricultural habitat that are more or less conducive to healthy plant growth.

37. The method of claim 26, further comprising:
each of said one or more of said sensor modules having an identify defined by a unique label;
a robotic system or a drone scanning said unique label; and
said robotic system or said drone using the sensor module identity to map a location of the module within the agricultural habitat.

38. The method of claim 26, further comprising:
positioning and repositioning said one or more sensor modules within the agricultural habitat with either lift rings or a ferromagnetic plate associated with the sensor module and with which the sensor module may be picked up and moved by a robot system.

39. The method of claim 38, wherein said lift rings further providing an insulated mount having either of electrical contacts or an inductive coupling mechanism by which the sensor module receives power to charge a sensor module energy source and by which the sensor module may be interrogated to receive information from, or output information to, an external system.
